(19) United States
(12) Patent Application Publication
(10) Pub. No.: US 2005/0277650 A1
(43) Pub. Date: Dec. 15, 2005

(54) Process for preparing aripirazole hydrate

(76) Inventors: Sundaram Venkataraman, Hyderabad (IN); Padi Pratap Reddy, Hyderabad (IN); Ballikonda Satyanarayana, Hyderabad (IN); Tamma Rangareddy, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/109,123

(22) Filed: Apr. 19, 2005

Related U.S. Application Data
(60) Provisional application No. 60/604,113, filed on Aug. 24, 2004.

(30) Foreign Application Priority Data
Apr. 20, 2004 (IN) 362/CHEM/2004

(51) Int. Cl. A61K 31/496; C07D 43/02

(52) U.S. Cl. 514/253.07; 544/363

(57) ABSTRACT

Aripriprazole hydrate is prepared by dissolving apripiprazole in a hydrous organic solvent at elevated temperature, adding seed crystals of aripiprazole hydrate to the solution, cooling the mixture, and isolating crystals of aripiprazole hydrate.

### CROSS-REFERENCE TO RELATED APPLICATIONS

This applications claims priority to copending U.S. Provisional Application 60/604,113 filed Aug. 24, 2004, and to India Patent Application 362/CHE/2004 filed Apr. 20, 2004. The entire content of each of these prior applications is hereby incorporated by this reference.

### INTRODUCTION TO THE INVENTION

The present invention relates to an improved process for the preparation of 7-[4-[4-(2,3-dichlorophenyl)-1-piperazinyl]butoxy]-3,4-dihydro-2-(1H)-quinolinone hydrate (aripiprazole hydrate). Aripiprazole has structural formula (1).

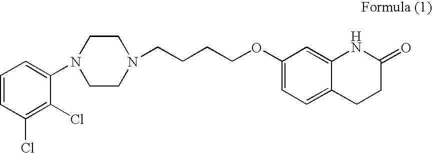

Aripiprazole is an atypical antipsychotic agent useful for the treatment of schizophrenia. Schizophrenia is a common type of psychosis characterized by delusions, hallucinations and extensive withdrawal from others. Onset of schizophrenia typically occurs between the age of 16 and 25 and affects 1 in 100 individuals worldwide. It is more prevalent than Alzheimer's disease, multiple sclerosis, insulin-dependent diabetes and muscular dystrophy. Early diagnosis and treatment can lead to singificantly improved recovery and outcome. Moreover, early therapeutic intervention can avert costly hospitalization.

U.S. Pat. No. 4,734,416 and U.S. Pat. No. 5,006,528 disclose arpiprazole and processes for the preparation thereof. The said patents also dislose various salts of aripiprazole and their preparation. Preparation of conventional apripiprazole hydrate was disclosed in Fourth Japanese-Korean symposium on Separaton Technology (Oct. 6-8, 1996) and WO 03/026659 teaches the preparation of apripiprazole hydrate by dissolving the aripirprazole crude crystals in a hydrous organic solvent, subsequently heating followed by cooling the resulting solution. The organic solvent should be one which is miscible with water, such as for example an alcohol, acetone, an ether or a mixture thereof, with ethanol being particularly desirable. The amount of water in the hydrous solvent can be 10-25% by volume of the solvent, or preferably close to 20% by volume.

It has been obseved that aripiprazole hydrate prepared as per WO 03/026659 is not getting consistent results.

Accordingly, a feature of the present invention is giving a simple and easy handling process for the preparation of aripiprazole hydrate with consistent results.

### SUMMARY OF THE INVENTION

The present invention provides a process for preparing aripiprazole hydrate comprising:

i. dissolving aripiprazole in an organic solvent, containing water;

ii. heating the solution from step (i) to temperatures above about 67° C.;

iii. seeding the solution of step (ii) with aripiprazole hydrate crystals at temperatures above about 67° C.;

iv. cooling the solution from step (iii) to about 50° C. to about 55° C. and maintaining the temperatures as crystals form;

v. cooling the solution from step (iv) to about 0 to about 10° C. and maintaining the temperatures as crystals form;

vi. separating crystals from the solution; and

vii. drying the crystals at about 45 to about 50° C. until the water content is about 3 to about 4.5 percent by weight.

### BRIEF DESCRIPTION OF THE DRAWINGS

FIG. 1 is a Differential Scanning Calorimetry (DSC) thermogram of the present aripiprazole hydrate, obtained on a SHIMADZU-50 with two crucibles under an inert atmosphere, temperature up to 200° C. and 5.0° C. per min.

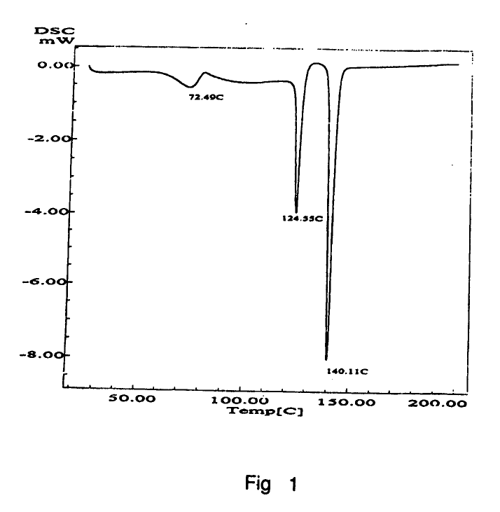

FIG. 2 is an X-ray powder diffractogram (XRD) of the aripiprazole hydrate, measured on Bruker Axe, DS Advance Powder X-ray diffractometer with Cu K alpha-1 radiation source.

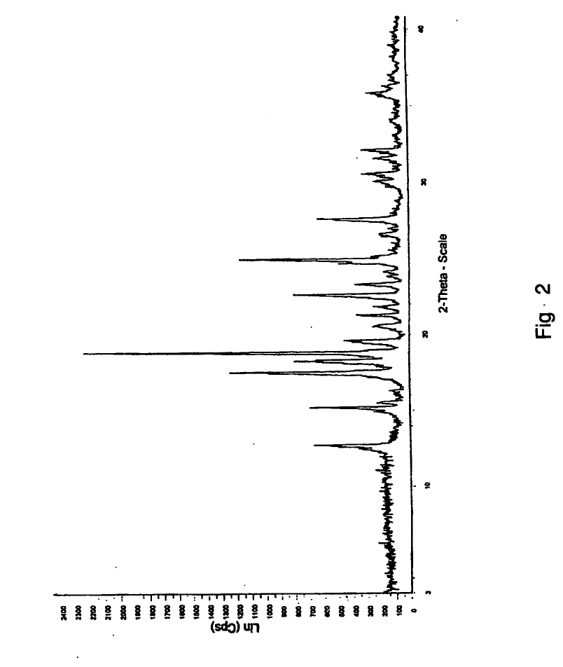

### DETAILED DESCRIPTION

The inventors of the present invention have discovered that seeding results in obtaining the desired aripiprazole hydrate in a consistent manner. The present invention provides an improved process for the preparation of aripiprazole hydrate.

The aripiprazole hydrate obtained by the process of the present invention can be identified by its DSC thermogram.

In one embodiment, an improved process for the preparation of aripiprazole hydrate of the present invention comprises:
i. dissolving aripiprazole in hydrous organic solvent, containing water, with heating;
ii. optionally, charging 5%-10% decolorizing carbon to the clear solution of step (i) and stirring at the heating temperature for 15-60 minutes;
iii. removal of the carbon under the hot condition;
iv. heating the filtrate of step (iii) under stirring to about 67° C. to about 80° C.;
v. seeding the solution of step (iv) with aripiprazole hydrate at above 67° C.;
vi. stirring the reaction solution of step (v) at about 65-70° C.;
vii. cooling the reaction mass of step (vi) at about 50° C. to about 55° C. and maintaining at the same temperature for crystal formation;
viii. cooling the reaction mass of step (vii) further to about 0-10° C. and maintaining for crystal formation;
ix. filtration of the separated solid in step (viii) at about 0° C. to about 5° C. and washing the solid with chilled hydrous organic solvent; and
x. drying the solid of step (ix) at about 45-50° C. until the water content is about 4.5% w/w.

Preferably the hydrous organic solvents that can be used for the preparation of aripiprazole hydrate are alcohols such as methanol, ethanol, isopropanol, n-butanol and pentanol; or acetic acid, or tetrahydrofuran, or mixtures of any two or more thereof. The organic solvent preferably is isopropanol.

Preferably, the hydrous organic solvent contains from 5 to 30%, more preferably 15 to 25%, even more preferably about 20% of water.

Seeding the solution of step (iv) comprises adding aripiprazole hydrate crystals in an amount about 2% to 10% by weight of the dissolved aripiprazole, preferably about 5% by weight. The addition of seeding is carried out at temperatures above 67° C., preferably at about 70-80° C.

Aripiprazole hydrate prepared according to the present invention has a mean particle size between 1 and 200 μm.

An improved process of the present invention is a simple, cost effective, industrially scaleable and environment friendly process for the synthesis of aripiprazole hydrate.

The process of the present invention will be explained in more detail with reference to the following examples, which are provided by way of illustration only and should not be construed as limit to the scope of the claims in any manner.

### REFERENCE EXAMPLE

Preparation of Crude Aripiprazole

1-(2,3-Dichlorophenyl)-piperazinyl. HCl (74 gm, 0.2767 moles), methanol (375 ml) and triethyl amine (77 ml, 0.55 moles) were charged and the mixture was stirred 5-10 minutes at 25-35° C. 7-(4-Bromobutoxy)-3,4-dihydro-2-(1H)-quinolinone (70 gm, 0.25 moles) was added and reaction mixture was heated to reflux temperature and maintained for 30 hours for reaction completion. Isolated solid was filtered at 50-55° C. and washed with methanol (75 ml). Wet compound and methanol (2250 ml) were charged into an autoclave vessel, then maintained at 80-85° C. for 60-90 minutes. The reaction mass was cooled to 25-35° C. and maintained for 60-90 minutes. Solid was filtered and washed with methanol (75 ml). Wet compound was charged into water (750 ml) and stirred for 2-3 hours at 25-35° C. The compound was filtered and washed with water (75 ml). Product was dried at about 60-70° C. until a constant weight was obtained. (Yield 72.7%)

### Example 1

Preparation of Aripiprazole Hydrate

Aripiprazole crude (70 g, prepared according to the reference example) was refluxed in 20% aqueous isopropanol (1400 ml) and stirred for complete dissolution. Decolorizing carbon (7 grams) was added and the mixture was maintained at reflux for 15-30 minutes, then the mixture was filtered while hot under vacuum on indiflow perlite fillter aid grade 1-100 and washed with 20% aqueous isopropanol (35 ml). The filtrate were heated to reflux and maintained at reflux for 10-20 minutes, then seeding material (aripiprazole hydrate 3.5 gm) was added at 73-75° C. Maintained the reaction mass at 65-70° C. for 45-60 minutes and maintained at 50-55° C. for a further period of 45-60 minutes. Reaction mass was cooled to 25-35° C. then further cooled to 0-5° C. Maintained at 0-5° C. for 30-60 minutes. The product was filtered and washed with 20% aqueous isopropanol (70 ml). Product was dried at 45-50° C. for 1-2 hours. (Yield 98.3%, HPLC purity 99.8%)

The particle size was measured by the Malvern particle size analyzer, which shows the aripiprazole hydrate has the particle size 1-150 microns.

### Example 2

Preparation of Aripiprazole Anhydride

Aripiprazole hydrate of example 1 (56.0 gm) was dried at 78-82° C. in a tray drier for 12-15 hours till the moisture content was below 0.5% w/w to obtain aripiprazole anhydride. (yield 98.4%, hplc purity 99.8%)

1. A process for preparing aripiprazole hydrate comprising:
i. dissolving aripiprazole in an organic solvent, containing water;
ii. heating the solution from step (i) to temperatures above about 67° C.;
iii. seeding the solution of step (ii) with aripiprazole hydrate crystals at temperatures above about 67° C.;
iv. cooling the solution from step (iii) to about 50° C. to about 55° C. and maintaining the temperatures as crystals form;
v. cooling the solution from step (iv) to about 0 to about 10° C. and maintaining the temperatures as crystals form;
vi. separating crystals from the solution; and
vii. drying the crystals at about 45 to about 50° C. until the water content is about 3 to about 4.5 percent by weight.

2. The process according to claim 1, wherein the hydrous organic solvent comprises an alcohol, acetic acid, or tetrahydrofuran.

3. The process according to claim 1, wherein the hydrous organic solvent comprises methanol, ethanol, isopropanol, n-butanol or pentanol.

4. The process according to claim 1, wherein the hydrous organic solvent comprises isopropanol.

5. The process according to claim 1, wherein the hydrous organic solvent contains about 5 to about 30 percent by weight water.

6. The process according to claim 1, wherein the hydrous organic solvent contains about 15 to about 25 percent by weight water.

7. The process according to claim 1, wherein the hydrous organic solvent comprises isopropanol having about 20 percent by weight of water.

8. The process according to claim 1, wherein seeding is carried out at temperatures about 70° C. to about 80° C.

9. The process according to claim 1,wherein seeding comprises adding aripiprazole hydrate crystals in amounts about 2 to about 10 percent by weight of the original aripiprazole.

10. The process according to claim 1, wherein seeding comprises adding aripiprazole hydrate crystals in amounts about 5% by weight of the original aripiprazole.

11. The process according to claim 1, further comprising the step of mixing carbon with the solution of step (i) at an elevated temperature and then removing the carbon before conducting step (iii).

12. The process according to claim 1, wherein the dried crystals have a mean particle size between about 1 μm and about 150 μm

13. A process for preparing aripiprazole hydrate comprising:
i dissolving aripiprazole in an organic solvent comprising isopropanol and containing about 20 percent by weight water;
ii heating the solution from step (i) to reflux and adding carbon;
iii removing the carbon from the solution;
iv at temperatures at least about 67° C., seeding the solution of step (iii) with aripiprazole hydrate crystals;
v cooling the solution from step (iv) to about 50° C. to about 55° C. and maintaining the temperatures as crystals form;
vi cooling the solution from step (v) to about 0° C. to about 10° C. and maintaining the temperatures as crystals form;
vii separating crystals from the solution; and
viii. drying the crystals at about 45° C. to about 50° C. until the water content is about 3 to about 4.5 percent by weight.
